SEQUENCE LISTING (18 sequences)

SEQ ID NO: 1 - protein, 75 aa, synthetic construct
  REGION 1..75: note = Synthetic: Complete Hila sequence
NECIRKWLSCVDRKNDCCEGLECYKRRHSFEVCVPIPGFCLVKWKQCDGRERDCCAGLECWKRSGNKSSVCAPIT

SEQ ID NO: 2 - protein, 34 aa, synthetic construct
  REGION 1..34: note = Synthetic: Hila N Lobe sequence
NECIRKWLSCVDRKNDCCEGLECYKRRHSFEVCV

SEQ ID NO: 3 - protein, 38 aa, synthetic construct
  REGION 1..38: note = Synthetic: Hila C Lobe sequence
GFCLVKWKQCDGRERDCCAGLECWKRSGNKSSVCAPIT

SEQ ID NO: 4 - skipped

SEQ ID NO: 5 - protein, 32 aa, synthetic construct
  REGION 1..32: note = Synthetic: Hila C Lobe fragment
CLVKWKQCDGRERDCCAGLECWKRSGNKSSVC

SEQ ID NO: 6 - protein, 32 aa, synthetic construct
  REGION 1..32: note = Synthetic: Hila C Lobe fragment with S27R mutation
CLVKWKQCDGRERDCCAGLECWKRRGNKSSVC

SEQ ID NO: 7 - protein, 33 aa, synthetic construct
  REGION 1..33: note = Synthetic: Hila C Lobe fragment with S27R mutation
CLVKWKQCDGRERDCCAGLECWKRRGNKSSVCA

SEQ ID NO: 8 - protein, 34 aa, synthetic construct
  REGION 1..34: note = Synthetic: Hila C Lobe fragment with S27R mutation
CLVKWKQCDGRERDCCAGLECWKRRGNKSSVCAP

SEQ ID NO: 9 - protein, 35 aa, synthetic construct
  REGION 1..35: note = Synthetic: Hila C Lobe fragment with S27R mutation
CLVKWKQCDGRERDCCAGLECWKRRGNKSSVCAPI

SEQ ID NO: 10 - protein, 36 aa, synthetic construct
  REGION 1..36: note = Synthetic: Hila C Lobe fragment with S27R mutation
CLVKWKQCDGRERDCCAGLECWKRRGNKSSVCAPIT

SEQ ID NO: 11 - protein, 33 aa, synthetic construct
  REGION 1..33: note = Synthetic: Hila C Lobe fragment with S27R mutation
FCLVKWKQCDGRERDCCAGLECWKRRGNKSSVC

SEQ ID NO: 12 - protein, 34 aa, synthetic construct
  REGION 1..34: note = Synthetic: Hila C Lobe fragment with S27R mutation
FCLVKWKQCDGRERDCCAGLECWKRRGNKSSVCA

SEQ ID NO: 13 - protein, 35 aa, synthetic construct
  REGION 1..35: note = Synthetic: Hila C Lobe fragment with S27R mutation
FCLVKWKQCDGRERDCCAGLECWKRRGNKSSVCAP

SEQ ID NO: 14 - protein, 36 aa, synthetic construct
  REGION 1..36: note = Synthetic: Hila C Lobe fragment with S27R mutation
FCLVKWKQCDGRERDCCAGLECWKRRGNKSSVCAPI

SEQ ID NO: 15 - protein, 37 aa, synthetic construct
  REGION 1..37: note = Synthetic: Hila C Lobe with S27R mutation
FCLVKWKQCDGRERDCCAGLECWKRRGNKSSVCAPIT

SEQ ID NO: 16 - protein, 75 aa, synthetic construct; identical to SEQ ID NO: 1
  REGION 1..75: note = Synthetic: complete Hila with S27R mutation

SEQ ID NO: 17 - protein, 32 aa, synthetic construct
  REGION 1..32: note = Synthetic: Hila C Lobe fragment with G28R mutation
CLVKWKQCDGRERDCCAGLECWKRSRNKSSVC

SEQ ID NO: 18 - protein, 32 aa, synthetic construct
  REGION 1..32: note = Synthetic: Hila C Lobe fragment with N29S mutation
CLVKWKQCDGRERDCCAGLECWKRSGSKSSVC

(19) United States
(12) Patent Application Publication
(10) Pub. No.: US 2026/0285919 A1
(43) Pub. Date: Sep. 24, 2026

(54) RECOMBINANT PEPTIDES AND COMPOSITIONS THEREOF FOR PAIN MANAGEMENT

(71) Applicant: UNIVERSITY OF ROCHESTER, Rochester, NY (US)

(72) Inventors: David MACLEAN, Rochester, NY (US); Kyle BERGER, Macedon, NY (US)

(21) Appl. No.: 19/474,450

(22) PCT Filed: Jun. 11, 2024
(86) PCT No.: PCT/US24/33424

§ 371 Date: Oct. 10, 2025

Related U.S. Application Data
(60) Provisional application No. 63/508,097, filed on Jun. 14, 2023.

(51) Int. Cl. C07K 14/435; A61K 9/00; A61K 38/00; A61K 45/06; A61K 47/54; A61P 29/00; C12N 15/86

(52) U.S. Cl. CPC C07K 14/43518; A61K 9/0019; A61K 45/06; A61K 47/542; A61P 29/00; C12N 15/86; A61K 38/00

(57) ABSTRACT

A recombinant peptide comprising a modified human Hila C lobe sequence, wherein the peptide inhibits an activity of human ASIC1b receptors. A method for treating pain in a subject, comprising the step of administering to the subject an effective amount of a pharmaceutical composition comprising the recombinant peptide.

This application is a national stage application of International Application No. PCT/US2024/033424, filed Jun. 11, 2024, which claims priority of U.S. Provisional Application No. 63/508,097 filed on Jun. 14, 2023. The entirety of all the aforementioned applications is incorporated herein by reference.

This invention was made with government support under MH125135 and DE021985 awarded by the National Institutes of Health. The government has certain rights in the invention.

### FIELD

This application relates generally to the field of medicine, and in particular, to the control of pain and pain management.

### BACKGROUND

ASICs were discovered in the late 1990s, almost 20 years after the observation that sensory neurons depolarize in response to a sudden drop in pH. Although they belong to the epithelial sodium channel/degenerin family of receptors, they are distinguished by their predominantly neuronal distribution and activation by decreases in extracellular pH. Alternative splicing of four ASIC-encoding genes leads to the expression of six subunits (ASIC1a, ASIC1b, ASIC2a, ASIC2b, ASIC3 and ASIC4) that combine to form hetero- or homo-trimeric channels that differ in their pH sensitivity, kinetics, and susceptibility to desensitization.

A specific peptide (mambalgin) inhibits both ASIC1a and ASIC1b when injected into the paws of mice. Pain responses in these injected paws are found to be much less sensitive. However, when a toxin called PcTx1, which only blocks ASIC1a, is injected then this effect is not seen, suggesting the mambalgin effect is not through inhibition of ASIC 1a, but through ASIC1b. Consistent with this, in mice where ASIC1a is genetically deleted, the mambalgin toxin still works since ASIC1b is present. This indicates that the peripheral analgesia effect is through ASIC1b, not ASIC1a.

Pain studies are nearly always begun in rodents before moving to other organisms then ultimately humans for clinical trials. However, the human ASIC1b protein and the rodent ASIC1b proteins are different; consequently, the mambalgin toxin actually increases the activity of human ASIC1b.

There remains a need for a high affinity ASIC1b inhibitor in humans that can reduce pain sensitivity.

### SUMMARY

One aspect of the application is a recombinant peptide comprising a modified human Hila C lobe sequence, wherein the peptide inhibits the activity of human ASIC1b receptors.

In some embodiments, the modified Hila C lobe sequence comprises the amino acid residues of CLVKWKQCDGRERDCCAGLECWKR₂₆S₂₇G₂₈N₂₉KSSVC with one or more amino acid substitutions at amino acid residues R₂₆, S₂₇, G₂₈, and/or N₂₉.

In some embodiments, the amino acid substitutions are one or more of R₂₆X₁, S₂₇X₂, G₂₈X₃ and N₂₉X₄, wherein X₁ is selected from the group consisting of A, N, D, C, Q, E, G, H, I, L, K, M, F, P, S, T, W,Y,V; X₂ is selected from the group consisting of A, N, D, C, Q, E, G, H, I, L, K, M, F, P, R, T, W,Y,V; X₃ is selected from the group consisting of A, N, D, C, Q, E, R, H, I, L, K, M, F, P, S, T, W, Y, V; and X₄ is selected from the group consisting of A, R, D, C, Q, E, G, H, I, L, K, M, F, P, S, T, W, Y, V. In some embodiments, X₂ is R. In some embodiments, X₃ is R. In some embodiments, X₄ is S.

In some embodiments, the modified Hila C lobe sequence comprises the sequence of SEQ ID NO:5.

In some embodiments, the modified Hila C lobe sequence comprises the sequence of anyone of SEQ ID NOS: 6-15.

In some embodiments, the modified Hila C lobe sequence comprises the sequence of SEQ ID NO:16, SEQ ID NO:17 or SEQ ID NO:18.

Another aspect of the present application relates to a peptide-conjugate comprising (1) a recombinant peptide of the present application and (2) a conjugation partner.

In some embodiments, the conjugation partner comprises one or more duration enhancing moieties selected from the group consisting of polyethylene glycol and long chain acyl fatty acids. In some embodiments, the conjugation partner is linked to the recombinant peptide through a linker.

Another aspect of the present application relates to a polynucleotide encoding a recombinant peptide of the present application.

An aspect of the present application relates to an expression vector comprising a polynucleotide of the present application and a regulatory element operably linked to the polynucleotide. In some embodiments, the expression vector is a viral vector.

An aspect of the present application relates to a pharmaceutical composition, comprising: (1) a recombinant peptide of the present application, or a peptide conjugate of the present application, or an expression vector of the present application; and (2) a pharmaceutically acceptable carrier.

An aspect of the present application relates to a method for treating pain in a subject, comprising the step of administering to the subject an effective amount of the pharmaceutical composition of the present application. In some embodiments, the pharmaceutical composition is administered locally by intramuscular injection. In some embodiments, the pharmaceutical composition is administered systemically by intravenous injection.

In some embodiments, the method further comprises the step of administering to the subject an additional agent. In some embodiments, the additional agent is an inhibitor of ASIC1a. In some embodiments, the additional agent is an inhibitor of ASIC3.

### BRIEF DESCRIPTION OF THE DRAWINGS

FIG. 1 shows sequence (upper) and structure (lower) of Hila protein. The circle illustrates the S₂₇ position.

FIG. 2 shows the Hila C-lobe peptide with an S27R substitution inhibits human ASIC1b receptors. Data obtained from expressing human ASIC1b in recombinant cell line and measuring receptor activity in the presence of S27R using electrophysiology.

While the present disclosure will now be described in detail, and it is done so in connection with the illustrative embodiments, it is not limited by the particular embodiments illustrated in the figures and the appended claims.

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan.

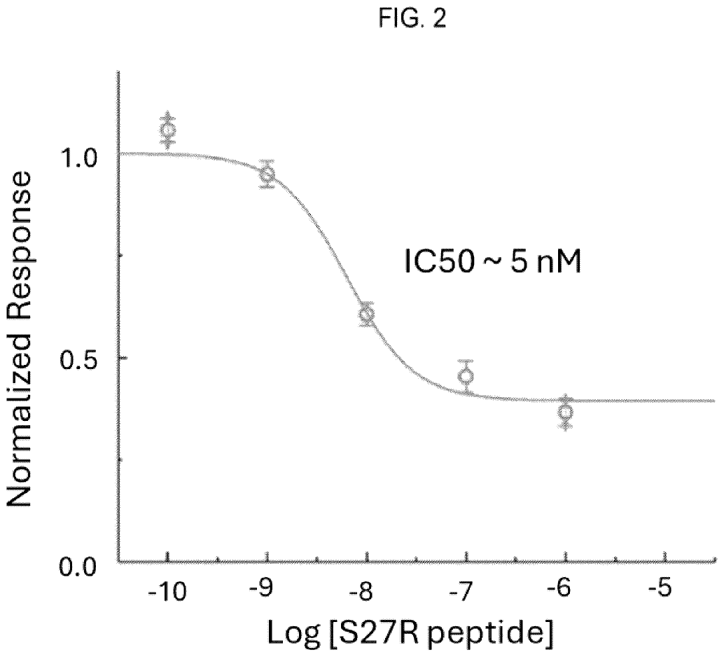

This application is directed to methods of pain treatment by using an ASIC1 peptide inhibitor called Hila, first reported herein. Hila is made of two ‘lobes’ called N and C. FIG. 2 shows the full Hila toxin with the two lobes colored in blue or red as well as the amino acid sequences.

The present teachings provide a system, including methods and compositions, for treatment of pain. The methods may include approaches for reducing injury resulting from pain and/or for identifying drugs for pain treatment. The methods selectively may inhibit one or more members of the acid sensing ion channel (ASIC) family, to provide a targeted therapy for pain treatment.

### I. Definitions

The term “ASIC1b inhibitor composition” and “pharmaceutical composition” are used herein with reference to a composition comprising ASIC1b inhibitor and at least one pharmaceutically acceptable carrier. When referring to these compositions with regard to dosages, the weights are given in terms of the amount by weight of ASIC1b inhibitor.

A “functional homologue” or a “functional equivalent” of a given polypeptide includes molecules derived from the native polypeptide sequence, as well as recombinantly produced or chemically synthesized polypeptides which function in a manner similar to the reference molecule to achieve a desired result. As used herein, the term “functionally active” means retaining at least 50, 55, 60, 65, 70, 75, 80, 85, 90, 95, 96, 97, 98, 99 or 100% of the ASIC1b inhibitive activity of the peptide of SEQ ID NO:3.

A “variant” of a particular polypeptide or polynucleotide has one or more additions, substitutions, and/or deletions with respect to the polypeptide or polynucleotide, which may be referred to as the “original polypeptide” or “original polynucleotide”, respectively. An addition may be an insertion or may be at either terminus. A variant may be shorter or longer than the original polypeptide or polynucleotide. The term “variant” encompasses “fragments”.

A “fragment” is a continuous portion of a polypeptide or polynucleotide that is shorter than the original polypeptide. In some embodiments a variant comprises or consists of a fragment. In some embodiments a fragment or variant is at least 20%, 30%, 40%, 50%, 60%, 70%, 80%, 90%, 95%, 96%, 97%, 98%, 99%, or more as long as the original polypeptide or polynucleotide. A fragment may be an N-terminal, C-terminal, or internal fragment. In some embodiments a variant polypeptide comprises or consists of at least one domain of an original polypeptide.

A “derivative” of a peptide of the present application is a peptide that has been altered, for example by conjugation or complexing with a chemical moiety, by post-translational modification (including, but not limited to, phosphorylation, glycosylation, acetylation, lipidation or pegylation), or by the addition of one or more amino acids (including, for example, the addition of a protein or tag to assist with purification). The incorporation of non-naturally occurring amino acids is also encompassed by “derivative”. The addition or incorporation of other chemical or physical groups such as fluorescent compounds, radioisotopes, or spin labels is also encompassed by “derivative”. In some embodiments, the functionally active variant or derivative has a half-maximal inhibitory concentration (IC₅₀) for inhibiting ASIC1b of less than about 50 nM. In some embodiments, the functionally active variant or derivative has a half-maximal inhibitory concentration (ICso) for inhibiting ASIC1b of less than about 10 nM.

The term “conservative” amino acid substitution as used herein to refer to amino acid substitutions that substitute functionally-equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. The largest sets of conservative amino acid substitutions include:
(1) hydrophobic: His, Tip, Tyr, Phe, Met, Leu, He, Val, Ala;
(2) neutral hydrophilic: Cys, Ser, Thr;
(3) polar: Ser, Thr, Asn, Gin;
(4) acidic/negatively charged: Asp, Glu; (5) charged: Asp, Glu, Arg, Lys, His;
(5) basic/positively charged: Arg, Lys, His;
(6) basic: Asn, Gin, His, Lys, Arg;
(7) residues that influence chain orientation: Gly, Pro; and
(8) aromatic: Trp, Tyr, Phe, His.

In addition, “structurally-similar” amino acids can substitute conservatively for some of the specific amino acids. Groups of structurally-similar amino acids include: (He, Leu, and Val); (Phe and Tyr); (Lys and Arg); (Gin and Asn); (Asp and Glu); and (Gly and Ala). In this regard, it is understood that amino acids are substituted on the basis of side-chain bulk, charge, and/or hydrophobicity. Amino acid residues are classified into four major groups:

Acidic: The residue has a negative charge due to loss of an H ion at physiological pH and the residue is attracted by aqueous solution so as to seek the surface positions in the conformation of a peptide in which it is contained when the peptide is in aqueous solution.

Basic: The residue has a positive charge due to association with an Hion at physiological pH and the residue is attracted by aqueous solution so as to seek the surface positions in the conformation of a peptide in which it is contained when the peptide is in aqueous medium at physiological pH.

Neutral/non-polar: The residues are not charged at physiological pH and the residue is repelled by aqueous solution so as to seek the inner positions in the conformation of a peptide in which it is contained when the peptide is in aqueous medium. These residues are also designated “hydrophobic residues.”

Neutral/polar: The residues are not charged at physiological pH, but the residue is attracted by aqueous solution so as to seek the outer positions in the conformation of a peptide in which it is contained when the peptide is in aqueous medium. “Amino acid” residues can be further classified as cyclic or non-cyclic, and aromatic or non-aromatic with respect to their side-chain groups, these designations being commonplace to the skilled artisan.

The term “subject” as used herein, means a human or a non-human mammal, including but not limited to a dog, cat, horse, donkey, mule, cow, domestic buffalo, camel, llama, alpaca, bison, yak, goat, sheep, pig, elk, deer, domestic antelope, or a non-human primate selected for treatment or therapy.

A “subject in need thereof' means a subject identified as in need of a therapy or treatment.

A “therapeutic effect” relieves, to some extent, one or more of the symptoms of a disease or condition. “Curing” means that the symptoms of active disease or condition are eliminated. However, certain long-term or permanent effects of the disease may exist even after a cure is obtained (such as tissue damage and the like).

The phrase “therapeutically effective amount” as used herein refers to an amount of ASIC1b inhibitor that ameliorates, attenuates, or eliminates one or more of the symptoms of a particular disease or condition or prevents, modifies, or delays the onset of one or more of the symptoms of a disease or condition.

“Treat”, “treatment,” and “treating,” as used herein, refer to administering an ASIC1b inhibitor composition for prophylactic and/or therapeutic purposes. The term “prophylactic treatment” refers to treating a patient who does not yet have the relevant disease or condition, but who is susceptible to, or otherwise at risk of, a particular disease or condition, whereby the treatment reduces the likelihood that the patient will develop the disease or condition. The term “therapeutic treatment” refers to administering treatment to a patient already having a disease or condition.

“Preventing” or “prevention” refers to delaying or forestalling the onset, development or progression of a disease or condition for a period, including weeks, months, or years.

“Amelioration” means a lessening of severity of at least one indicator of a disease or condition. In certain embodiments, amelioration includes a delay or slowing in the progression of one or more indicators of a disease or condition. The severity of indicators may be determined by subjective or objective measures which are known to those skilled in the art.

“Administering” means providing a pharmaceutical agent or composition to a subject, and includes, but is not limited to, administering by a medical professional and self-administering.

Administration of the ASIC1b inhibitor compositions of the present application can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly.

“Parenteral administration,” means administration through injection or infusion.

Parenteral administration includes, but is not limited to, subcutaneous administration, intravenous administration, intramuscular administration, intraarterial administration, and intracranial administration.

“Pharmaceutical composition” means a mixture of substances suitable for administering to an individual that includes a pharmaceutical agent. For example, a pharmaceutical composition may comprise a modified oligonucleotide and a sterile aqueous solution.

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable carrier” or “pharmaceutically acceptable excipient” includes all solvents, diluents, emulsifiers, binders, buffers, dispersion media, coatings, antibacterial and antifungal agents, isotonic and absorption delaying agents and the like, or any other such compound as is known by those of skill in the art to be useful in preparing pharmaceutical formulations. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

A “unit dosage form” refers to a composition containing an amount of a compound that is suitable for administration to a subject, in a single dose, according to good medical practice. However, as further described below, the preparation of a single or unit dosage form, however, does not imply that the dosage form is administered once per day or once per course of therapy.

An “effective amount” or “effective dose” of an agent (or composition containing such agent) refers to the amount sufficient to achieve a desired biological and/or pharmacological effect, e.g., when delivered to a cell or organism according to a selected administration form, route, and/or schedule. As will be appreciated by those of ordinary skill in this art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered to a subject in a single dose, or through use of multiple doses, in various embodiments.

As used herein, the term “agent” is used with reference to any substance, compound (e.g., molecule), supramolecular complex, material, or combination or mixture thereof. A compound may be any agent that can be represented by a chemical formula, chemical structure, or sequence. Example of agents, include, e.g., small molecules, polypeptides, nucleic acids (e.g., RNAi agents, antisense oligonucleotide, aptamers), lipids, polysaccharides, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells, to produce a biological effect. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

A nucleotide or amino acid residue in a first nucleic acid or protein “corresponds to” a residue in a second nucleic acid or protein if the two residues perform one or more corresponding functions and/or are located at corresponding positions in the first and second nucleic acids or proteins. Corresponding functions are typically the same, equivalent, or substantially equivalent functions, taking into account differences in the environments of the two nucleic acids or proteins as appropriate. Residues at corresponding positions typically align with each other when the sequences of the two nucleic acids or proteins are aligned to maximize identity (allowing the introduction of gaps) using a sequence alignment algorithm or computer program such as those referred to below (see “Identity”) and/or are located at positions such that when the 3-dimensional structures of the proteins is superimposed the residues overlap or occupy structurally equivalent positions and/or form the same, equivalent, or substantially equivalent intramolecular and/or intermolecular contacts or bonds (e.g., hydrogen bonds). The structures may be experimentally determined, e.g., by X-ray crystallography or NMR or predicted, e.g., using structure prediction or molecular modeling software. An alignment may be over the entire length of one or more of the aligned nucleic acid or polypeptide sequences or over at least one protein domain (or nucleotide sequence encoding a protein domain).

A “domain” of a protein is a distinct functional and/or structural unit of a protein, e.g., an independently folding unit of a polypeptide chain. In some embodiments a domain is a portion of a protein sequence identified as a domain in the Conserved Domain Database of the NCBI (Marchler-Bauer A et al. (2013), “CDD: conserved domains and protein three-dimensional structure”, Nucleic Acids Res. 41 (DI): D384-52). In some embodiments corresponding amino acids are the same in two sequences (e.g., a lysine residue, a threonine residue) or would be considered conservative substitutions for each other. Examples of corresponding residues include (i) the catalytic residues of two homologous enzymes and (ii) sites for post-translational modification of a particular type (e.g., phosphorylation) within corresponding structural or functional domains that have similar effects on the structure or function of homologous proteins.

“Homology” refers to the percent of identity between two polynucleotide or two polypeptide moieties. The correspondence between a polynucleotide sequence from one moiety to another can be determined by techniques known in the art. For example, homology can be determined by a direct comparison of the sequence information between two polypeptide molecules by aligning the sequence information and using readily available computer programs to evaluate the extent of homology. Alternatively, homology can be determined by hybridization of polynucleotides under conditions which allow for the formation of stable duplexes between homologous regions, followed by digestion with single stranded-specific nuclease(s), and size determination of the digested fragments. Two DNA, or two polypeptide sequences are “substantially homologous” to each other when at least about 80%, preferably at least about 90%, and most preferably at least about 95%, at least about 96%, at least about 97%, at least about 98%, or at least about 99% of the nucleotides or amino acids match over a defined length of the molecules, as determined using the methods above.

A “vector” is used herein with reference to a recombinant plasmid or virus that includes a heterologous nucleic acid of interest to be delivered into a host cell, either in vitro or in vivo. The nucleic acid of interest may be linked to, e.g., inserted into, the vector using, e.g., restriction and ligation. Vectors include, for example, DNA or RNA plasmids, cosmos, naturally occurring or modified viral genomes or portions thereof, nucleic acids that can be packaged into viral capsids, mini-chromosomes, artificial chromosomes, etc. Plasmid vectors typically include an origin of replication (e.g., for replication in prokaryotic cells). A plasmid may include part or all of a viral genome (e.g., a viral promoter, enhancer, processing or packaging signals, and/or sequences sufficient to give rise to a nucleic acid that can be integrated into the host cell genome and/or to give rise to infectious virus). Viruses or portions thereof that can be used to introduce nucleic acids into cells may be referred to as viral vectors, which are further described below. A vector may contain one or more nucleic acids encoding a marker suitable for identifying and/or selecting cells that have taken up the vector. Markers include, for example, various proteins that increase or decrease either resistance or sensitivity to antibiotics or other agents (e.g., a protein that confers resistance to an antibiotic such as puromycin, hygromycin or blasticidin), enzymes whose activities are detectable by assays known in the art (e.g., —galactosidase or alkaline phosphatase), and proteins or RNAs that detectably affect the phenotype of cells that express them (e.g., fluorescent proteins). Vectors often include one or more appropriately positioned sites for restriction enzymes, which may be used to facilitate insertion into the vector of a nucleic acid, e.g., a nucleic acid to be expressed.

An “expression vector” is a vector designed to incorporate a desired nucleic acid of interest in operable linkage to regulatory elements (also termed “regulatory sequences”, “expression control elements”, or “expression control sequences”) mediating expression of the nucleic acid of interest as an RNA transcript (e.g., an mRNA that can be translated into protein or a noncoding RNA such as an shRNA or miRNA precursor). Expression vectors include regulatory sequence(s), e.g., expression control sequences, sufficient to direct transcription of an operably linked nucleic acid under at least some conditions; other elements required or helpful for expression may be supplied by, e.g., the host cell or by an in vitro expression system. Such regulatory sequences typically include a promoter and may include enhancer sequences or upstream activator sequences. In some embodiments a vector may include sequences that encode a 5′ untranslated region and/or a 3′ untranslated region, which may comprise a cleavage and/or polyadenylation signal. In general, regulatory elements may be contained in a vector prior to insertion of a nucleic acid whose expression is desired or may be contained in an inserted nucleic acid or may be inserted into a vector following insertion of a nucleic acid whose expression is desired. Expression vectors include non-viral vectors such as plasmid vectors, and viral vectors such as adeno virus vectors, adeno-associated virus (AAV) vectors, lentivirus vectors, herpes virus vectors.

As used herein, a nucleic acid and regulatory element(s) are said to be “operably linked” when they are covalently linked so as to place the expression or transcription of the nucleic acid under the influence or control of the regulatory element(s). For example, a promoter region would be operably linked to a nucleic acid if the promoter region were capable of effecting transcription of that nucleic acid. One of ordinary skill in the art will be aware that the precise nature of the regulatory sequences useful for gene expression may vary between species or cell types, but may in general include, as appropriate, sequences involved with the initiation of transcription, RNA processing, or initiation of translation. The choice and design of an appropriate vector and regulatory element(s) is within the ability and discretion of one of ordinary skill in the art. For example, one of skill in the art will select an appropriate promoter (or other expression control sequences) for expression in a desired species (e.g., a mammalian species) or cell type. A vector may contain a promoter capable of directing expression in mammalian cells, such as a suitable viral promoter, e.g., from a cytomegalovirus (CMV), retrovirus, simian virus (e.g., SV40), papilloma virus, herpes virus or other virus that infects mammalian cells, or a mammalian promoter from, e.g., a gene such as globin, actin, phosphoglycerate kinase (PGK), etc., or a composite promoter such as a CAG promoter (combination of the CMV early enhancer element and chicken beta-actin promoter). In some embodiments a human promoter may be used. In some embodiments, a promoter that ordinarily directs transcription by a eukaryotic RNA polymerase II (a “pol II promoter”) or a functional variant thereof is used. In some embodiments, a promoter that ordinarily directs transcription by a eukaryotic RNA polymerase I promoter, e.g., a promoter for transcription of ribosomal RNA (other than 5S rRNA) or a functional variant thereof is used. In some embodiments, a promoter that ordinarily directs transcription by a eukaryotic RNA polymerase III (a “pol III promoter”), e.g., (a U6, HI, 7SK or tRNA promoter or a functional variant thereof) may be used. One of ordinary skill in the art will select an appropriate promoter for directing transcription of a sequence of interest. Examples of expression vectors that may be used in mammalian cells include, e.g., the pcDNA vector series, pSV2 vector series, pCMV vector series, pRSV vector series, pEFl vector series, Gateway® vectors, etc. In some embodiments, regulatable (e.g., inducible or repressible) expression control element(s), e.g., a regulatable promoter, is/are used so that expression can be regulated, e.g., turned on or increased or turned off or decreased. For example, the tetracycline-regulatable gene expression system can be employed to provide inducible or repressible expression. Other inducible/repressible systems may be used in various embodiments. For example, expression control elements that can be regulated by small molecules such as artificial or naturally occurring hormone receptor ligands (e.g., steroid receptor ligands), rapamycin, and metal ions may be used in certain embodiments.

### II. Recombinant Peptides

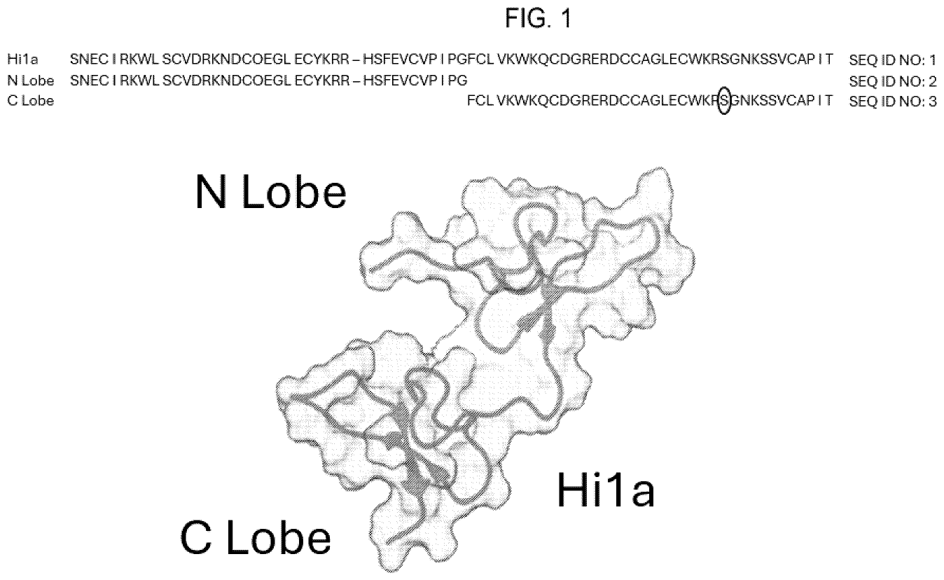

Hila is an 8.6-kDa peptide (SEQ ID NO:1) containing six disulfide bonds. Hi 1a is a double-knot toxin in which two inhibitor cystine knot (ICK) motifs, the N-lobe (SEQ ID NO: 2) and the C-lobe (SEQ ID NO:3), are joined head-to-tail by a three-residue linker (SEQ ID NO:4). The ICK motif is a common feature of spider-venom peptides, and it typically results in these peptides having high levels of chemical and thermal stability as well as resistance to proteases. A ribbon structure of Hila is shown in FIG. 1.

One aspect of the present application relates to a recombinant peptide comprising a modified Hila C lobe sequence, wherein the peptide inhibits the activity of human ASIC1b receptors. In some experiments, the activity is the ASIC channel activity measured by patch clamp electrophysiology where recombinant human ASIC1b is expressed in a mammalian cell line (this particular cell line lacks endogenous ASIC protein) and subjected to either whole cell patch clamp or outside out patch clamp recording. The human ASIC1b receptors are activated in the recorded cell by rapidly switching between extracellular buffers of differing pH, either with or without recombinant peptide.

In certain embodiments, the modified human Hila C lobe sequence comprises the amino acid residues of CLVKWKQCDGRERDCCAGLECWKR₂₆S₂₇G₂₈N₂₉KSSVC (SEQ ID NO:5) with one or more amino acid substitutions at amino acid residues R₂₆, S₂₇, G₂₈, and/or N₂₉.

In certain embodiments, the amino acid substitutions are one or more of R₂₆X₁, S₂₇X₂, G₂₈X₃ and N₂₉X₄ of SEQ ID NO:1, wherein X₁ is selected from the group consisting of A, N, D, C, Q, E, G, H, I, L, K, M, F, P, S, T, W, Y, V; X₂ is selected from the group consisting of A, N, D, C, Q, E, G, H, I, L, K, M, F, P, R, T, W, Y, V; X₃ is selected from the group consisting of A, N, D, C, Q, E, R, H, I, L, K, M, F, P, S, T, W, Y, V; X₄ is selected from the group consisting of A, R, D, C, Q, E, G, H, I, L, K, M, F, P, S, T, W, Y, V.

In some embodiments of the recombinant peptide, wherein X₂ is R.

In some embodiments of the recombinant peptide, wherein X₃ is R.

In some embodiments of the recombinant peptide, wherein X₄ is S.

In certain embodiments, the peptide comprises the sequence of CLVKWKQCDGRERDCCAGLECWKRR₂₇GNKSSVC (SEQ ID NO:6).

In certain embodiments, the peptide comprises the sequence of CLVKWKQCDGRERDCCAGLECWKRR₂₇GNKSSVCA (SEQ ID NO:7).

In certain embodiments, the peptide comprises the sequence of CLVKWKQCDGRERDCCAGLECWKRR₂₇GNKSSVCAP (SEQ ID NO:8).

In certain embodiments, the peptide comprises the sequence of CLVKWKQCDGRERDCCAGLECWKRR₂₇GNKSSVCAPI (SEQ ID NO:9).

In certain embodiments, the peptide comprises the sequence of CLVKWKQCDGRERDCCAGLECWKRR₂₇GNKSSVCAPIT (SEQ ID NO:10).

In certain embodiments, the peptide comprises the sequence of FCLVKWKQCDGRERDCCAGLECWKRR₂₇GNKSSVC (SEQ ID NO:11).

In certain embodiments, the peptide comprises the sequence of FCLVKWKQCDGRERDCCAGLECWKRR₂₇GNKSSVCA (SEQ ID NO:12).

In certain embodiments, the peptide comprises the sequence of FCLVKWKQCDGRERDCCAGLECWKRR₂₇GNKSSVCAP (SEQ ID NO:13).

In certain embodiments, the peptide comprises the sequence of FCLVKWKQCDGRERDCCAGLECWKRR₂₇GNKSSVCAPI (SEQ ID NO:14).

In certain embodiments, the peptide comprises the sequence of FCLVKWKQCDGRERDCCAGLECWKRR₂₇GNKSSVCAPIT (SEQ ID NO:15).

In certain embodiments, the peptide comprises the sequence of NECIRKWLSCVDRKNDCCEGLECYKRRHSFEVCVPIPGFCLVKWKQCDGRERDCCA GLECWKRR₂₇GNKSSVCAPIT (SEQ ID NO:16).

In certain embodiments, the peptide comprises the sequence of CLVKWKQCDGRERDCCAGLECWKRSR₂₈NKSSVC (SEQ ID NO:17).

In certain embodiments, the peptide comprises the sequence of CLVKWKQCDGRERDCCAGLECWKRSGS₂₉KSSVC (SEQ ID NO:18).

In some embodiments, the peptide further comprises one or more conservative amino acid substitutions at positions other than R₂₆S₂₇G₂₈N₂₉.

In some embodiments, the peptide further comprises one or more amino acid substitutions with non-standard and/or non-naturally occurring amino acids. Such substitutions can include, but are not necessarily limited to, (1) non-standard positively charged amino acids, like: ornithine; N-(4-aminobutyl)-glycine having a lysine side chain attached to the “N-terminus” and aminopropyl or aminoethyl groups attached to the amino group of glycine; (2) Non-naturally occurring amino acids with no net charge and sidechains similar to arginine, such as citrulline, with or without methylene groups; (3) non-standard non-naturally occurring amino acids with OH (e.g., serine), such as, homoserine, hydroxyproline, hydroxyvaline, and penicillamin; (4) praline derivatives, such as, D-Pro, including 3,4-dehydroproline, pyroglutamine, praline with fluorine substitutions on the ring, 1,3-thiazolidine-4-carboxylic acid; (5) Histidine derivative, such as beta-(2-thienyl)-alanine; or (6)alkyl derivatives, such as 2-aminobutyric acid, norvaline, norleucine, homoleucine, and alpha-aminoisobutyric acid.

In some embodiments, the peptide may further contain one or more covalently attached functional groups, preferably attached to either one or both of the N and C termini of the peptide. The peptide modifications can be used e.g., to increase peptide stability, in vivo half-life, solubility and facilitate attachment of proteinaceous or non-proteinaceous moieties.

Exemplary N-terminal modification include acetylation, biotinylation, dansyl labelling, fluorescein-labelling, 7-methoxycoumarin acetic acid (Mca)-labelling, palmitic acid conjugation, methylation (i.e., —NHCH₃ or —NH (CH₃)₂), adding a 1-amino-cyclohexane-carboxylic acid moiety (Chex); and adding a carbobenzoyl group, or blocking the amino terminus with any blocking group containing a carboxylate functionality defined by RCOO—, where R is selected from the group consisting of naphthyl, acridinyl, steroidyl, and similar groups. An exemplary C-terminal modification is amidation. Where the C-terminus is amidated, the carboxylic acid of the amino acid is converted to an amide, i.e., NH₂—CH₂—C(O)—NH₂.

Additional modifications include carboxylation, glycosylation, methylation (e.g., substitution of a-hydrogens with methyl groups), carbonylation, phosphorylation, dimerization, addition of interchain and/or intrachain disulfide bonds, addition of trans olefin, derivatization by known protecting/blocking groups, circularization, and substitution with D amino acids.

Covalently attached groups can include stabilizers, couplers, ligands, enzymatic substrates and/or combinations thereof. Preferred groups include acyl groups on the N terminus and cysteamine (cya) coupling groups on the C terminal end. To the latter may be conveniently attached other chemical moieties, e.g., dyes, ligands, proteins, enzymes, enzymatic substrates, etc. Alternatives to cya are also known to those of skill in the art. For stabilizing and/or blocking, e.g., cya may be replaced with an alky group such as methyl or ethyl, which are known to be conveniently positioned onto a—COOH group.

A derivitizing group, including, but not limited to, a sulfhydryl-containing group or moiety may be positioned at the C-terminus of the peptide, even when it is not coupled to another chemical moiety. In one embodiment, the C-terminal end may be modified with a cysteamide group (—NH—CH₂—CH₂—SH), which can allow further coupling to drugs. A. cysteamide group is compatible with the peptide synthesis using the Fmoc strategy and leads to a C-terminal protected peptide. Alternatively, the peptide can include a C-terminal cysteine residue containing a sulfuydryl (—SH) group that can be optionally utilized for conjugation to other moieties. In another embodiment, the C-terminal end includes a 2,4-diamino-butyric acid (DAB) moiety. C-terminal modifications may further include replacing the free acid with a carboxamide group or forming a cyclic lactam at the carboxy terminus to introduce structural constraints.

Naturally occurring side chains of the 20 genetically encoded amino acids (or D amino acids) may be replaced with other side chains with similar properties, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclic.

In another embodiment, the C-terminal carboxyl group or a C-terminal ester may be induced to cyclize by internal displacement of the —OH or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Such methods are well known in the art.

In other embodiments, the recombinant peptide of the present invention is cyclized or includes a desamino or descarboxy residue at the peptide termini so that there are no terminal amino or carboxyl groups. This can decrease susceptibility to proteases and/or to restrict the conformation of the peptide. C-terminal functional groups of the compounds of the present invention include amide, amide lower alkyl, amide di(lower alkyl), lower alkoxy, hydroxy, and carboxy, and the lower ester derivatives thereof, and the pharmaceutically acceptable salts thereof. The recombinant peptide may be cyclized by adding an N and/or C terminal cysteine and cyclizing the peptide through disulfide linkages or other side chain interactions.

In some embodiments, the recombinant peptides of the present invention are synthesized using traditional liquid- or solid-phase synthesis. Fmoc and t-Boc solid phase peptide synthesis (SPPS) can be employed to grow the peptides from carboxy to amino-terminus.

In certain embodiments, the recombinant peptides further comprises an additional amino acid sequence that confers inhibitory effect on ASIC1a. In some embodiments, the additional amino acid sequence comprises SEQ ID NO:2.

### III. Peptide-Conjugates

Another aspect of the present application relates to a peptide-conjugate comprising (1) the recombinant peptide of the present application; and (2) a conjugation partner.

In some embodiments, conjugation partner comprises one or more duration enhancing moieties that improves the circulating life, water solubility and/or antigenicity of administered proteins,

In some embodiments, the linked duration enhancing moiety includes a polyethylene glycol. Polyethylene glycol (“PEG”) has been used in efforts to obtain therapeutically usable peptides. As appreciated by one of skill in the art, the PEG backbone [(CH₂CH₂—C)—)n, n: number of repeating monomers] is flexible and amphiphilic. Without wishing to be bound by any theory or mechanism of action, the long, chain-like PEG molecule or moiety is believed to be heavily hydrated and in rapid motion when in an aqueous medium. This rapid motion is believed to cause the PEG to sweep out a large volume and prevents the approach and interference of other molecules. As a result, when attached to another chemical entity (such as a peptide), PEG polymer chains can protect such chemical entity from immune response and other clearance mechanisms. As a result, pegylation can lead to improved drug efficacy and safety by optimizing pharmacokinetics, increasing bioavailability, and decreasing immunogenicity and dosing frequency. “Pegylation” refers to conjugation of a PEG moiety with another compound. Unless expressly indicated to the contrary, the terms “PEG,” “polyethylene glycol polymer” and the like refer to polyethylene glycol polymer and derivatives thereof, including methoxy-PEG (mPEG).

Methods for attaching polymer moieties, such as PEG and related polymers, to reactive groups found on a peptides and proteins are well known in the art. Typical attachment sites in proteins include primary amino groups, such as those on lysine residues or at the N-terminus, thiol groups, such as those on cysteine side-chains, and carboxyl groups, such as those on glutamate or aspartate residues or at the C-terminus. Common sites of attachment are to the sugar residues of glycoproteins, cysteines or to the N-terminus and lysines of the target peptide. The terms “pegylated” and the like refer to covalent attachment of polyethylene glycol to a peptide or other biomolecule, optionally through a linker as described herein and/or as known in the art.

In some embodiments, a PEG moiety in a peptide conjugate described herein has a nominal molecular weight within a specified range. The size of a PEG moiety is indicated by reference to the nominal molecular weight, typically provided in kilodaltons (kD). The molecular weight is calculated in a variety of ways known in the art, including number, weight, viscosity and “Z” average molecular weight. It is understood that polymers, such as PEG and the like, exist as a distribution of molecule weights about a nominal average value.

Reference to PEGs of other molecular weights follows this convention. In some embodiments, the PEG moiety has a nominal molecular weight in the range 10-100 KD, 20-80 KD, 20-60 KD, or 20-40 KD. In some embodiments, the PEG moiety has a nominal molecular weight of 10, 15, 20, 25, 30, 35, 40, 45, 50, 55, 60, 65, 70, 75, 80, 85, 90, 95 or even 100 KD.

Preferably, the PEG moiety has a molecular weight of 20, 25, 30, 40, 60 or 80 KD.

PEG molecules useful for derivatization of peptides are typically classified into linear, branched and Warwick (i.e., PolyPEG®) classes of PEGs, as known in the art. Unless expressly indicated to the contrary, the PEG moieties described herein are linear PEGs.

Furthermore, the terms “two arm branched,” “Y-shaped” and the like refer to branched PEG moieties, as known in the art. The term “Warwick” in the context of PEGs, also known as “comb” or “comb-type” PEGs, refers to a variety of multi-arm PEGs attached to a backbone, typically poly(methacrylate), as known in the art.

In some embodiments, the conjugation partner is a duration-enhancing conjugation partner that comprises one or more hydrophobic moieties chemically conjugated to the recombinant peptide of the present application. The hydrophobic moieties may be conjugated to N-terminus or C-terminus of the recombinant peptide through any free functional group on the N-terminal or C-terminal amino acids, for example, to the 8-amino group of lysine. Conjugation of the hydrophobic moiety can enhance anti-fusogenic activity of the recombinant peptide so that the activity is significantly higher after conjugation than prior to conjugation.

The hydrophobic moiety can be a fatty acid, sterol or fat soluble vitamin, such as vitamin A, vitamin D, vitamin E or vitamin K. The fatty acid can be a saturated, unsaturated, monounsaturated, or polyunsaturated fatty acid. In some embodiments, the fatty acid for conjugation is a beta-hydroxy fatty acid or a beta-amino fatty acid. In other embodiments, the fatty acids is selected from the group consisting of decanoic acid, undecanoic acid, dodecanoic acid, myristic acid, palmitic acid, stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, and cerebronic acid.

In some embodiments, the hydrophobic moiety is a sterol, such as a steroid with a hydroxyl group at the 3-position of the A-ring. Non-limiting examples of sterols for conjugation include, but are not limited to, zoosterols, such as cholesterol or derivatives thereof; and phytosterols, such as stigmasterol, beta-sitosterol, campesterol, ergosterol (provitamin D2), brassicasterol, delta-7-stigmasterol and delta-7-avenasterol.

In some embodiments, the hydrophobic moiety is an aliphatic group comprising between 6-22 carbons and a reactive group through which the aliphatic group is linked to the peptide. Non limiting examples of the reactive groups include, but are not limited to a carboxyl group, a carbonyl group, an amine group, a thiol group, a maleimide, an imido ester, an N-hydroxysuccinimide, alkyl halide, and aryl azide.

In some embodiments, the conjugation partner is attached to the recombinant peptide of the present application via linkers known in the art. In some embodiments, the conjugation partner has the formula of —L—R, wherein R is a duration enhancing moiety as described herein, and L is a linker or a bond. Where L is a linker, L can be —C(O)—, —NH—, —O—, —S—, —S—S—, —OCO—, —OCONH—, —NHCONH—, substituted or unsubstituted alkylene, substituted or unsubstituted alkenylene, substituted or unsubstituted urethane, substituted or unsubstituted alkylamide, substituted or unsubstituted alkylsulfone, substituted or unsubstituted heteroalkylene, substituted or unsubstituted cycloalkylene, substituted or unsubstituted heterocycloalkylene, substituted or unsubstituted arylene, or substituted or unsubstituted heteroarylene, and the like, as known in the art.

In some embodiments, L is R¹-substituted or unsubstituted alkylene, R¹-substituted or unsubstituted alkenylene, R¹-substituted or unsubstituted urethane, R¹-substituted or unsubstituted alkylamide, R¹-substituted or unsubstituted alkylsulfone, R¹-substituted or unsubstituted heteroalkylene, R¹-substituted or unsubstituted cycloalkylene, R¹-substituted or unsubstituted heterocycloalkylene, substituted or unsubstituted arylene, or substituted or unsubstituted heteroarylene. R¹ is R²-substituted or unsubstituted alkyl, R²-substituted or unsubstituted heteroalkyl, R²-substituted or unsubstituted cycloalkyl, R²-substituted or unsubstituted heterocycloalkyl, R²-substituted or unsubstituted aryl, or R²-substituted or unsubstituted heteroaryl. R² is R³-substituted or unsubstituted alkyl, R³-substituted or unsubstituted heteroalkyl, R³-substituted or unsubstituted cycloalkyl, R³-substituted or unsubstituted heterocycloalkyl, R³-substituted or unsubstituted aryl, or R³-substituted or unsubstituted heteroaryl. R³ is unsubstituted alkyl, unsubstituted heteroalkyl, unsubstituted cycloalkyl, unsubstituted heterocycloalkyl, unsubstituted aryl or unsubstituted heteroaryl.

In some embodiments, the linked duration enhancing moiety-L—R is covalently bonded to an amino acid side chain of the recombinant peptide, or to a backbone atom or moiety thereof. Exemplary backbone moieties include a free amine at the N-terminal, and a free carboxyl or carboxylate at the C-terminal. In some embodiments, an amino acid side chain or a backbone atom or moiety is covalently bonded to a polyethylene glycol, a long chain aliphatic group, or a derivative thereof.

### IV. Polynucleotides

Another aspect of the present application relates to a polynucleotide encoding a recombinant peptide described herein.

Another aspect of the present application relates to an expression vector comprising: the polynucleotides described herein; and a regulatory element operably linked to the polynucleotide.

The terms “codon optimized” and “codon optimization” refer to a process for modifying a nucleic acid sequence according to one or more of the following: (1) to match codon frequencies in a host organism target; (2) to promote increased expression; (3) to ensure proper folding; (4) to provide a GC content suitable for increasing mRNA stability or reducing secondary structures; (5) to minimize tandem repeat codons or base runs that may impair gene construction or expression; (6) to customize transcriptional and translational control regions; (7) to insert or remove protein trafficking sequences; (8) to remove/add post translation modification sites in an encoded protein (e.g. glycosylation sites); (9) to add, remove or shuffle protein domains; (10) to insert or delete restriction sites; (11) modify ribosome binding sites and mRNA degradation sites; (12) to adjust translational rates to allow the various domains of the protein to fold properly; or (13) to reduce or eliminate problem secondary structures within the polynucleotide. Codon optimization tools, algorithms and services are known in the art-non-limiting examples include services from GeneArt (Life Technologies), DNA2.0 (Menlo Park Calif) and/or proprietary methods.

In certain embodiments, the polynucleotides of the present application are codon optimized. In certain embodiment, the nucleic acid is codon optimized for expression in humans. In certain embodiments, codon-optimized polynucleotides for use according to the present application are prepared by replacing the codons of the polynucleotide encoding an recombinant peptide as described herein with e.g., “humanized” codons (e.g., codons that appear frequently in highly expressed human genes). Codon optimization methods are known in the art and may be used as provided herein. In some embodiments, the open reading frame (ORF) sequence in a polynucleotide is optimized using optimization algorithms as described herein and known in the art.

In some embodiments, a codon optimized polynucleotide sequence shares less than 95%, less than 90%, less than 85%, less than 80%, less than 75%, less than 70%, less than 65%, less than 60%, less than 55% or less than 50% sequence identity to a naturally-occurring or wild-type sequence (e.g., a naturally-occurring or wild-type DNA or mRNA sequence encoding a Hila polypeptide of interest.

In some embodiments, a codon optimized polynucleotide sequence shares between 50% and 95%, between 50% and 90%, between 50% and 85%, between 50% and 80%, between 50% and 75%, between 50% and 70%, between 50% and 65%, between 50% and 60%, between 50% and 55%, between 55% and 95%, between 55% and 90%, between 55% and 85%, between 55% and 80%, between 55% and 75%, between 55% and 70%, between 55% and 65%, between 55% and 60%, between 60% and 95%, between 60% and 90%, between 60% and 85%, between 60% and 80%, between 60% and 75%, between 60% and 70%, between 60% and 65%, between 65% and 95%, between 65% and 90%, between 65% and 85%, between 65% and 80%, between 65% and 75%, between 65% and 70%, between 70% and 95%, between 70% and 90%, between 70% and 85%, between 70% and 80%, between 70% and 75%, between 75% and 95%, between 75% and 90%, between 75% and 85%, between 75% and 80%, between 80% and 95%, between 80% and 90%, between 80% and 85%, between 85% and 95%, between 85% and 90%, or between 90% and 95% sequence identity to a naturally-occurring or wild-type sequence (e.g., a naturally-occurring or wild-type DNA or mRNA sequence encoding a recombinant Hi la peptide sequence as set forth herein.

In certain preferred embodiments, a polynucleotide of the present application includes one or more codon optimized full-length or substantially full-length recombinant Hila nucleotide sequences encoding the recombinant peptides as set forth herein, or any combination thereof.

In certain embodiments, the expression vector is a viral vector.

As used herein, the term “viral vector” refers to a recombinant polynucleotide vector comprising virally-derived nucleic acids containing sequences facilitating replication and expression of exogenously incorporated transgene sequences operatively linked to suitable control elements and one or more heterologous sequences (i.e., nucleic acid sequence not of viral origin).

The choice and design of an appropriate vector and regulatory element(s) is within the ability and discretion of one of ordinary skill in the art. For example, one of ordinary skill in the art can select an appropriate promoter (or other expression control sequences) for expression in a desired species (e.g., a mammalian species) or cell type. A vector may contain a promoter capable of directing expression in mammalian cells, such as a suitable viral promoter, e.g., from a cytomegalovirus (CMV), retrovirus, simian virus (e.g., SV40), papilloma virus, herpes virus or other virus that infects mammalian cells, or a mammalian promoter from, e.g., a gene such as EFI, ubiquitin (e.g., ubiquitin B or C), globin, actin, phosphoglycerate kinase (PGK), etc., or a composite promoter such as a CAG promoter (combination of the CMV early enhancer element and chicken beta-actin promoter). In some embodiments a human promoter may be used. In some embodiments, a promoter that ordinarily directs transcription by a eukaryotic RNA polymerase II (a “pol II promoter”) or a functional variant thereof is used. In some embodiments, a promoter that ordinarily directs transcription by a eukaryotic RNA polymerase I promoter, e.g., a promoter for transcription of ribosomal RNA (other than 5S rRNA) or a functional variant thereof is used. In some embodiments, a promoter that ordinarily directs transcription by a eukaryotic RNA polymerase III (a “pol III promoter”), e.g., (a U6, HI, 7SK or tRNA promoter or a functional variant thereof) may be used.

Exemplary promoters include, but are not limited to, the cytomegalovirus (CMV) immediate early promoter, an RSV LTR, a MoMLV LTR, a phosphoglycerate kinase-I (PGK) promoter, a simian virus 40 (SV40) promoter, a CK6 promoter, a transthyretin promoter (TTR), a TK promoter, a tetracycline responsive promoter (TRE), an HBV promoter, an hAAT promoter, a neuron-selective promoter, such as the human synapsin promoter, a muscle-specific promoter, such as the human creatine kinase (MCK) promoter, a liver-specific promoter, such as the human phosphoenolpyruvate carboxykinase (PEPCK) promoter, a rhodopsin kinase promoter, an opsin promoter, a U6 promoter, an E2F promoter, a telomerase (hTERT) promoter, an HI promoter, a cytomegalovirus enhancer/chicken beta-actin/rabbit-globin promoter (CAG) promoter, an elongation factor I-alpha promoter (EFI-) promoter, a human beta-glucuronidase promoter, a chicken beta-actin (CBA) promoter, a retroviral Rous sarcoma virus (RSV) LTR promoter, a dihydrofolate reductase promoter, and a 13-actin promoter.

Examples of expression vectors that may be used in mammalian cells include, e.g., the pcDNA vector series, pSV2 vector series, pCMV vector series, pRSV vector series, pEFI vector series, Gateway® vectors, etc. In some embodiments, regulatable (e.g., inducible or repressible) expression control element(s), e.g., a regulatable promoter, is/are used so that expression can be regulated, e.g., turned on or increased or turned off or decreased. For example, the tetracycline-regulatable gene expression system or variants thereof can be employed to provide inducible or repressible expression. Other inducible/repressible systems may be used in various embodiments. For example, expression control elements that can be regulated by small molecules such as artificial or naturally occurring hormone receptor ligands (e.g., steroid receptor ligands such as estrogen receptor or glucocorticoid receptor ligands), repamycin, and metal ions may be used in certain embodiments.

In some embodiments, one or more promoters are selected for expression of heterologous nucleic acids in a cell of the CNS to treat a disorder or disease of the CNS or PNS. In some embodiments, the promoter drives expression of the active agents in a brain cell. A brain cell may refer to any brain cell known in the art, including without limitation a neuron (such as a sensory neuron, motor neuron, interneuron, dopaminergic neuron, medium spiny neuron, cholinergic neuron, GABAergic neuron, pyramidal neuron, etc.), a glial cell (such as microglia, macroglia, astrocytes, oligodendrocytes, ependymal cells, radial glia, etc.), a brain parenchyma cell, and/or a Purkinje cell. In some embodiments, the neuron is a medium spiny neuron of the caudate nucleus, a medium spiny neuron of the putamen, a neuron of the cortex layer IV and/or a neuron of the cortex layer V.

CNS-selective or CNS-specific regulatory elements for CNS cells, brain cells, neurons, and glial cells are known in the art and include promoter-regulatory elements corresponding to neuron-specific enolase (NSE), myelin basic protein (MBP), glial fibrillary acid protein (GFAP), platelet-derived growth factor (PDGF), platelet-derived growth factor B-chain (PDGF-), synapsin (Syn), methyl-CpG binding protein 2 (MeCP2), Ca²⁺/calmodulin-dependent protein kinase II (CaMKII), metabotropic glutamate receptor 2 (mGluR2), neurofilament light (NFL) or heavy (NFH), —globin minigene n 2, preproenkephalin (PPE), enkephalin (Enk) and excitatory amino acid transporter 2 (EAAT2) promoters. Non-limiting examples of tissue-selective or tissue-specific expression elements for astrocytes include glial fibrillary acidic protein (GFAP) and EAAT2 promoters. A non-limiting example of a tissue-specific expression element for oligodendrocytes is the myelin basic protein (MBP) promoter.

In some embodiments, one or more promoters are selected for driving expression of the active agents in a muscle cell to treat a disorder or disease affecting muscle cells. Non-limiting examples of muscle-selective or muscle-specific promoters include mammalian muscle creatine kinase (MCK) promoters, mammalian desmin (DES) promoters, mammalian troponin I (TNNI2) promoters, and mammalian skeletal alpha-actin (ASKA) promoters.

### V. Pharmaceutical Compositions

Another aspect of the application relates to a pharmaceutical composition comprising: (1) the recombinant peptide described herein, or the peptide-conjugate described herein, or the expression vector described herein; and (2) a pharmaceutically acceptable carrier.

In some embodiments, the pharmaceutical composition further comprises an inhibitor of ASIC1a and/or ASIC3.

In some embodiments, the pharmaceutically acceptable carrier is a saline solution.

This can usefully be isotonic or hypotonic, particularly for pulmonary delivery. In some embodiments, the application also provides a delivery device (e.g. syringe, inhaler, nebuliser) which includes a pharmaceutical composition of the application.

The ASIC1b inhibitor pharmaceutical composition of the application is formulated to be compatible with its intended route of administration. Examples of routes of administration include parenteral, e.g., intrathecal, intra-arterial, intravenous, intradermal, subcutaneous, oral, transdermal (topical) and transmucosal administration.

Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose, pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor ELTM (BASF, Parsippany, NJ.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene, glycol, and liquid polyethylene glycol, and the like), and suitable mixtures thereof. The proper fluidity can be maintained, for example, using a coating such as lecithin, by the maintenance of the required particle size in the case of dispersion and using surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating an active agent in the required amount in an appropriate solvent with one or a combination of ingredients enumerated above, as required, followed by filtered sterilization. Dispersions can be prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral therapeutic administration, the active compound may be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished using nasal sprays or suppositories. For transdermal administration, the pharmaceutical compositions are formulated into ointments, salves, gels, or creams as generally known in the art.

In certain embodiments, the pharmaceutical composition is formulated for sustained or controlled release of the active ingredient. Biodegradable, biocompatible polymers can be used, such as ethylene vinyl acetate, polyanhydrides, polyglycolic acid, collagen, polyorthoesters, and poly lactic acid. Methods for preparation of such formulations will be apparent to those skilled in the art. The materials can also be obtained commercially from e.g., Alza Corporation and Nova Pharmaceuticals, Inc. Liposomal suspensions (including liposomes targeted to infected cells with monoclonal antibodies to viral antigens) can also be used as pharmaceutically acceptable carriers.

It is especially advantageous to formulate oral or parenteral compositions in dosage unit form for ease of administration and uniformity of dosage. Suitable unit dosage forms include, but are not limited to powders, tablets, pills, capsules, lozenges, suppositories, patches, nasal sprays, injectables, implantable sustained-release formulations, lipid complexes, etc.

A dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the present application is dictated by and directly dependent on the unique characteristics of the active compound and the particular therapeutic effect to be achieved, and the limitations inherent in the art of compounding such an active compound for the treatment of individuals.

Toxicity and therapeutic efficacy of the ASIC1b inhibitor composition of the present application can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. ASIC1b inhibitor compounds exhibiting large therapeutic indices are preferred.

While compounds that exhibit toxic side effects may be used, care should be taken to design a delivery system that targets such compounds to the site of affected tissue to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the present application, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to determine useful doses more accurately in humans. The pharmaceutical compositions can be included in a container, pack, or dispenser together with instructions for administration.

### VI. Method of Using the Recombinant Peptide of the Present Application

Another aspect of the present application relates to a method for treatment of a disease or condition in a subject, wherein the disease or condition is caused by ASIC1b activity or contributed by ASIC1b activity. The method comprises the step of administering a therapeutically effective amount of at least one recombinant peptide of the present application, a functional variant thereof or a functional derivative thereof, to the subject, wherein the recombinant peptide is capable of specifically binding to acid sensing ion channel subtype 1b (ASIC1b) and inhibiting the activity of ASIC1b.

Another aspect of the application relates to a method for treating pain in a subject, comprising the step of administering to the subject an effective amount of the pharmaceutical composition described herein.

In certain embodiments, the pharmaceutical composition is administered locally by intramuscular injection.

In certain embodiments, the pharmaceutical composition is administered systemically by intravenous injection.

In certain embodiments, the method further comprises the step of administering to the subject an additional agent.

In certain embodiments, the additional agent is an inhibitor of ASIC1a.

In certain embodiments, the additional agent is an inhibitor of ASIC3.

Administration (or administering), as used herein, includes any route of subject exposure to an inhibitor, under any suitable conditions, and at any suitable time(s).

Administration may be self-administration or administration by another, such as a health-care practitioner (e.g., a doctor, a nurse, etc.). Administration may be by injection (e.g., intravenous, intramuscular, subcutaneous, intracerebral, epidural, and/or intrathecal, among others), ingestion (e.g., using a capsule, lozenge, a fluid composition, etc.), inhalation (e.g., an aerosol (less than about 10 microns average droplet diameter) inhaled nasally and/or orally), absorption through the skin (e.g., with a skin patch) and/or mucosally (e.g., through oral, nasal, and/or pulmonary mucosa, among others), and/or the like. Mucosal administration may be achieved, for example, using a spray (such as a nasal spray), an aerosol that is inhaled), and/or the like. A spray may be a surface spray (droplets on average greater than about 50 microns in diameter) and/or a space spray (droplets on average about 10-50 microns in diameter).

A therapeutically effective amount of the recombinant peptide of the present application may be administered. A therapeutically effective amount of the recombinant peptide, as used herein, is any amount of the recombinant peptide that, when administered to subjects, reduces, in a significant number of the subjects, the degree, incidence, and/or extent of pain-induced injury in the subjects. Accordingly, a therapeutically effective amount may be determined, for example, in clinical studies in which various amounts of the recombinant peptide are administered to test subjects (and, generally, compared to a control group of subjects).

The recombinant peptide may be administered in any suitable form and in any suitable composition to subjects. In some examples, the recombinant peptide may be configured as a pharmaceutically acceptable salt. The composition may be formulated to include, for example, a fluid carrier/solvent (a vehicle), a preservative, one or more excipients, a coloring agent, a flavoring agent, a salt(s), an anti-foaming agent, and/or the like. The inhibitor may be present at a concentration in the vehicle that provides a therapeutically effective amount of the recombinant peptide for treatment of pain when administered to a subject in need thereof.

As a general proposition, the therapeutically effective amount of the recombinant peptide administered will be in a weight range of about 1 ng/kg body weight/day to about 100 mg/kg body weight/day whether by one or more administrations. In more particular embodiments, the recombinant peptide is administered in weight range from about 1 ng/kg body weight/day to about 1 μg/kg body weight/day, 1 ng/kg body weight/day to about 100 ng/kg body weight/day, 1 ng/kg body weight/day to about 10 ng/kg body weight/day, 10 ng/kg body weight/day to about 1 μg/kg body weight/day, 10 ng/kg body weight/day to about 100 ng/kg body weight/day, 100 ng/kg body weight/day to about 1 μg/kg body weight/day, 100 ng/kg body weight/day to about 10 μg/kg body weight/day, 1 μg/kg body weight/day to about 10 μg/kg body weight/day, 1 μg/kg body weight/day to about 100 μg/kg body weight/day, 10 μg/kg body weight/day to about 100 μg/kg body weight/day, 10 μg/kg body weight/day to about 1 mg/kg body weight/day, 100 μg/kg body weight/day to about 10 mg/kg body weight/day, 1 mg/kg body weight/day to about 100 mg/kg body weight/day and 10 mg/kg body weight/day to about 100 mg/kg body weight/day.

In other embodiments, the recombinant peptide is administered at a dosage range of 1 ng-10 ng per injection, 10 ng-100 ng per injection, 100 ng-1 μg per injection, 1 μg-10 μg per injection, 10 μg-100 μg per injection, 100 μg-1 mg per injection, 1 mg-10 mg per injection, 10 mg-100 mg per injection, and 100 mg-1000 mg per injection. The recombinant peptide may be injected once daily, twice daily, three times daily, and/or every 2, 3, 4, 5, 6 or 7 days. In addition, the recombinant peptide may be administered over a period of one month, two months, six months, 12 months, 2 years, 5 years, 10 years, 20 years, or more.

In other embodiments, the recombinant peptide may be administered in a range from about 1 ng/kg to about 100 mg/kg. In more particular embodiments, the recombinant peptide may be administered in a range from about 1 ng/kg to about 10 ng/kg, about 10 ng/kg to about 100 ng/kg, about 100 ng/kg to about 1 μg/kg, about 1 μg/kg to about 10 μg/kg, about 10 μg/kg to about 100 μg/kg, about 100 μg/kg to about 1 mg/kg, about 1 mg/kg to about 10 mg/kg, about 10 mg/kg to about 100 mg/kg, about 0.5 mg/kg to about 30 mg/kg, and about 1 mg/kg to about 15 mg/kg.

In other particular embodiments, the amount of the recombinant peptide administered is, or is about, 0.0006, 0.001, 0.003, 0.006, 0.01, 0.03, 0.06, 0.1, 0.3, 0.6, 1, 3, 6, 10, 30, 60, 100, 300, 600 and 1000 mg/day.

The specific dose of the recombinant peptide may be determined based on the particular circumstances of the individual patient including the size, weight, age and sex of the patient, the nature and stage of the disease, the aggressiveness of the disease, and the route of administration of the recombinant peptide.

In other embodiments, recombinant peptide of the present application is prescribed to be taken in combination with one or more other analgesic agents. When used in such combinations, the analgesic of the present application and other analgesic agents may be administered simultaneously, by the same or different routes, or at various times during treatment. Examples of other analgesic agents include, but are not limited to, lidocaine, bupivacaine, articaine, morphine, hydrocodone, oxycodone, buprenorphine, methadone, fentanyl, acetaminophen, ibuprofen, acetylsalicylic acid, or other non-steroidal anti-inflammatory drugs (NSAIDs).

The present application is further illustrated by the following examples that should not be construed as limiting. The contents of all references, patents, and published patent applications cited throughout this application, as well as the Figures and Tables, are incorporated herein by reference.

### EXAMPLES

### Example 1: Materials and Methods

Human Embryonic Kidney 293T cells with the endogenous ASIC1 gene deleted via CRISPR were used for all electrophysiology experiments. These HEK293T cells were maintained in MEM supplemented with 10% equifetal bovine serum (Atlas Biologicals) and PenStrep (Gibco) and passaged every 3-4 days but not more than 25 passages. Cells were plated on 35 mm tissue cultured treated petri dishes and transfected 1-2 days later using polyethylenimine 25k (Polysciences, Inc) with a mass ratio of 1:3 (cDNA: PEI). A pcDNA3. L (+) vector containing the sequence for human ASIC1b was used for all experiments unless otherwise indicated. Mutations to this, or toxin expression constructs, were introduced using site directed mutagenesis PCR and confirmed by Sanger sequencing. Electrophysiology recordings were performed 1-3 days post-transfection using standard methods. Briefly, outside-out patch or whole cell recordings were obtained using borosilicate patch pipettes with resistances of 3-6 MQ when filled with an internal pipette solution. The internal pipette solution was (in mM) 135 CsF, 11 EGTA, 10 HEPES, 10 MES, 2 MgCl₂, 1 CaCl₂), and pH adjusted to 7.4 using CsOH. External solutions were comprised of (in mM) 150 NaCl, 1 CaCl₂), 1 MgCl₂, and either 10 HEPES (pH 7.45) or 10 MES (pH 6 or less) and adjusted using Tris-base to the indicated pH. Data were acquired at 20-50 kHz and filtered online at 10 kHz using Clampexl 1, an Axopatch 200B amplifier and a 1550 converter (all Molecular Devices) at room temperature and with a holding potential of −60 mV. Series resistance was routinely compensated by 90-95% when the peak amplitude exceeded 100 pA. A home built double or triple barrel perfusion pipette (Vitrocom) attached to a piezo translator (Physik Instrumente) under computer control was used for fast perfusion. Piezo voltage commands were generally filtered between 50 and 100 Hz. The pLicC-MBP-APETx2 vector was purchased from AddGene. The coding sequence of Hila was custom synthesized and cloned in place of APETx2 within the pLicC-MBP-APETx2 vector purchased from AddGene. The MalEss signal sequence was removed and the resulting vector transformed into SHuffle cells, grown at 37° C. until the OD₆₀₀ reached approximately 1.0 then expression was induced with 0.1 mM IPTG. Bacterial cultures expressing MBP-Hila were grown out overnight at 20° C. and pelleted. Bacterial pellets were resuspended in lysis buffer and then sonicated. Cell lysates were passed through a gravity Ni-NTA column and then eluted with an Imidazole elution buffer. The eluate was incubated with TEV protein overnight to cleave the hexaHisMBP from the Hila or modified toxin. Hi la was isolated using a C18 column on an Agilent HPLC.

### Example 2: Generation of Recombinant Peptides

A peptide containing the C-Lobe of Hila with a S27R substitution (SEQ ID NO:15) was generated by constructing an expression plasmid vector encoding the peptide. The peptide was expressed in bacteria, then purified using a combination of His-tag purification, elution and C18 column HPLC purification and polishing.

### Example 3: In Vitro Testing of the ASIC1b Inhibitory Activity of the Recombinant Peptides

The peptide generated in Example 2 was tested in vitro for its effect on ASIC1b channel activity. Briefly, peptide prepared in Example 2 is diluted into standard extracellular buffer for electrophysiology experiments. ASIC KO HEK cells were transfected with either human ASIC1a, human ASIClb, these same subunits with a c terminal GFP tag or co-transfected with GFP. The GFP acts as a transfection marker. 1-2 days post-transfection, patch clamp recordings of GFP+ cells were performed, either in whole cell recording mode or using outside out patch configuration. In either case, the cell/patch and receptors are continually perfused with extracellular buffer at pH 7.4 or 7.5, then rapidly switched to pH 5.5 buffer to activate the ASIC channels. After a baseline period, the extracellular pH 7.4 buffer is changed to the same solution but containing increasing concentrations of purified toxin. The presence of the toxin will reduce the functional response to pH 5.5 buffer in a concentration dependent way. From this concentration dependent reduction, an IC₅₀ is obtained by fitting to a standard logistic dose response curve function. Unexpectedly, it has been shown that the C lobe alone, containing the S27R mutation, results in a toxin which inhibits human ASIC1b with very high affinity (FIG. 2). This C lobe toxin, and mutations thereof, represents a new direction of analgesia treatment.

### Example 4: In Vivo Testing of the Analgesic Activity of the Recombinant Peptides in Mice

The recombinant peptide or a control peptide is injected into the forepaws of mice. The mice are then placed onto a hotplate at painfully hot temperature. Measurements are taken to determine how long it takes for the mice to withdraw or lift up their paw (aka withdrawal latency) from the hotplate. This is a widely used measurement of peripheral analgesia in rodents.

To test the peripheral analgesic effect of Hila C domain or other mutations thereof, we plan to use three behavioral paradigms. Following interplantar (i.pl) injections of vehicle or toxin, mice will be tested by either a hot plate withdrawal test, von Frey filaments or Hargreaves test.

The 52° C. hot plate test will be used as the initial test to determine if Hila C domain or other mutations thereof have an analgesic effect. An i.pl. injection of recombinant toxin or saline vehicle will be administered as 20 μL injection in the hind paw of an 8-11 week old C57Bl/6J mouse. The mouse will be held in a towel and the injected hind paw will be placed on the hot plate at varying times after the injection of recombinant toxin or saline vehicle. An optimal dose of recombinant toxin will be used to determine the duration of any effect. Both male and female mice will be tested at times ranging from 2 min to 2 h after the administration of recombinant toxin or saline vehicle. In all assays, males and females will be compared. To verify specificity, ASIC1 knock-out mice will be tested for baseline values and response to Hila C domain or other mutations in the optimal antinociceptive assay.

Mechanical allodynia will be measured in a chemically-induced model of neuropathy. Cisplatin (2.3 mg/kg, i.p.) will be administered on alternative days with lactated Ringer's solution on intervening days over a 9-day period as described previously. This approach results in mice having neuropathy. Mechanical allodynia will be measured on day 10 following an i.pl. injection of recombinant toxin or saline vehicle. Every 20 min, for a total of 80 min, the threshold for tactile allodynia will be measured using series of calibrated von Frey filaments possessing a bending force from 0.4 to 6 g until the threshold that induced paw withdrawal is found. The filaments will be applied in increasing strength. The threshold will be defined as two withdrawals per trial for the same filament weight. Uninjured mice do not respond with paw withdrawal with von Frey filaments ranging up to 6 g. The ipsilateral hind paw will receive saline and will serve to account for innate variability among mice. % allodynia=100×([mean paw withdrawal force (g) in control group−paw withdrawal for (g) of each mouse]/mean paw withdrawal force (g) in control group).

The Hargreaves test will assess thermal nociception using an infrared beam of light. Mice will be given an i.pl. injection of either recombinant toxin or saline vehicle in a hind paw. The mouse will be placed on a transparent glass surface positioned over the animal with an open end of the container in contact with the glass surface. Movement is limited, but the mouse is not restrained. The infrared light stimulus comes from below and passes through the glass table. When the mouse withdraws its hind paw, the stimulus is removed. Time to withdraw is recorded. A cut-off time of 20 sec will be used to ensure no damage to the hind paw. Temperatures range from 45° C. to 52° C.

For all experiments, there will be at least 10 mice in each condition for each assay. An assay will be repeated on multiple days. Lidocaine will be used as a positive control to determine each assay is functional. Estrogen cycles will be monitored in females.

LIST OF SEQUENCES

| SEQ ID | | |
|---|---|---|
| NO: | Description | Sequence |
| 1 | Complete Hi1a | NECIRKWLSCVDRKNDCCEGLECYKRRHSFEVCVPIPGFC |
| | sequence | LVKWKQCDGRERDCCAGLECWKRSGNKSSVCAPIT |
| 2 | Hi1a N Lobe | NECIRKWLSCVDRKNDCCEGLECYKRRHSFEVCV |
| | sequence | |
| 3 | Hi1a C Lobe | GFCLVKWKQCDGRERDCCAGLECWKRSGNKSSVCAPIT |
| | sequence | |
| 4 | Hi1a linker | PIP |
| | sequence | |
| 5 | Hi1a C Lobe | CLVKWKQCDGRERDCCAGLECWKRSGNKSSVC |
| | fragment | |
| 6 | Hi1a C Lobe | CLVKWKQCDGRERDCCAGLECWKRRGNKSSVC |
| | fragment with | |
| | S27R mutation | |
| 7 | Hi1a C Lobe | CLVKWKQCDGRERDCCAGLECWKRRGNKSSVCA |
| | fragment with | |
| | S27R mutation | |
| 8 | Hi1a C Lobe | CLVKWKQCDGRERDCCAGLECWKRRGNKSSVCAP |
| | fragment with | |
| | S27R mutation | |
| 9 | Hi1a C Lobe | CLVKWKQCDGRERDCCAGLECWKRRGNKSSVCAPI |
| | fragment with | |
| | S27R mutation | |
| 10 | Hi1a C Lobe | CLVKWKQCDGRERDCCAGLECWKRRGNKSSVCAPIT |
| | fragment with | |
| | S27R mutation | |
| 11 | Hi1a C Lobe | FCLVKWKQCDGRERDCCAGLECWKRRGNKSSVC |
| | fragment with | |
| | S27R mutation | |
| 12 | Hi1a C Lobe | FCLVKWKQCDGRERDCCAGLECWKRRGNKSSVCA |
| | fragment with | |
| | S27R mutation | |
| 13 | Hi1a C Lobe | FCLVKWKQCDGRERDCCAGLECWKRRGNKSSVCAP |
| | fragment with | |
| | S27R mutation | |
| 14 | Hi1a C Lobe | FCLVKWKQCDGRERDCCAGLECWKRRGNKSSVCAPI |
| | fragment with | |
| | S27R mutation | |
| 15 | Hi1a C Lobe | FCLVKWKQCDGRERDCCAGLECWKRRGNKSSVCAPIT |
| | with S27R | |
| | mutation | |
| 16 | complete Hi1a | NECIRKWLSCVDRKNDCCEGLECYKRRHSFEVCVPIPGFC |
| | with S27R | LVKWKQCDGRERDCCAGLECWKRRGNKSSVCAPIT |
| | mutation | |
| 17 | Hi1a C Lobe | CLVKWKQCDGRERDCCAGLECWKRSRNKSSVC |
| | fragment with | |
| | G28R mutation | |
| 18 | Hi1a C Lobe | CLVKWKQCDGRERDCCAGLECWKRSGSKSSVC |
| | fragment with | |
| | N29S mutation | |

### Reference to Electronic Sequence Listing

The application contains a Sequence Listing which has been submitted electronically in .XML format and is hereby incorporated by reference in its entirety. Said. XML copy, created on Oct. 9, 2025, is named “1134-263 NATL .xml” and is 23,343 bytes in size. The sequence listing contained in this .XML file is part of the specification and is hereby incorporated by reference herein in its entirety.

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

1. A recombinant peptide comprising a modified Hila C lobe sequence, wherein the peptide inhibits an activity of human ASIC1b receptors.

2. The recombinant peptide of claim 1, wherein the modified Hila C lobe sequence comprises the amino acid residues of SEQ ID NO:5 (CLVKWKQCDGRERDCCAGLECWKR₂₆S₂₇G₂₈N₂₉KSSVC) with one or more amino acid substitutions at amino acid residues R₂₆, S₂₇, G₂₈, and/or N₂₉.

3. The recombinant peptide of claim 2, wherein the amino acid substitutions are one or more of R₂₆X₁, S₂₇X₂, G₂₈X₃ and N₂₉X₄, wherein
X₁ is selected from the group consisting of A, N, D, C, Q, E, G, H, I, L, K, M, F, P, S, T,W,Y,V;
X₂ is selected from the group consisting of A, N, D, C, Q, E, G, H, I, L, K, M, F, P, R, T,W,Y,V;
X₃ is selected from the group consisting of A, N, D, C, Q, E, R, H, I, L, K, M, F, P, S, T, W, Y, V; and
X₄ is selected from the group consisting of A, R, D, C, Q, E, G, H, I, L, K, M, F, P, S, T, W, Y, V.

4. The recombinant peptide of claim 3, wherein X₂ is R.

5. The recombinant peptide of claim 3, wherein X₃ is R.

6. The recombinant peptide of claim 3, wherein X₄ is S.

7. The recombinant peptide of claim 1, comprising the sequence of SEQ ID NO: 5.

8. The recombinant peptide of claim 2, comprising the sequence of anyone of SEQ ID NOS: 6-18.

9.-11. (canceled)

12. A peptide-conjugate comprising
(1) the recombinant peptide of claim 1; and
(2) a conjugation partner.

13. The peptide conjugate of claim 12, wherein said conjugation partner comprises one or more duration enhancing moieties selected from the group consisting of polyethylene glycol and long chain acyl fatty acids.

14. The peptide conjugate of claim 12, wherein the conjugation partner is linked to the recombinant peptide through a linker.

15. A polynucleotide encoding the recombinant peptide of claim 1.

16. An expression vector comprising:
the polynucleotide of claim 15; and
a regulatory element operably linked to the polynucleotide.

17. The expression vector of claim 16, wherein the expression vector is a viral vector.

18. A pharmaceutical composition, comprising:
(1) a recombinant peptide comprising a modified Hila C lobe sequence, or a peptide conjugate comprising the recombinant peptide comprising a modified Hila C lobe sequence and a conjugation partner, or an expression vector encoding the recombinant peptide comprising a modified Hila C lobe sequence; and
(2) a pharmaceutically acceptable carrier,
wherein the recombinant peptide inhibits an activity of human ASIC1b receptors.

19. A method for treating pain in a subject, comprising the step of administering to the subject an effective amount of the pharmaceutical composition of claim 18.

20. The method of claim 19, wherein the pharmaceutical composition is administered locally by intramuscular injection or wherein the pharmaceutical composition is administered systemically by intravenous injection.

21. (canceled)

22. The method of claim 19, further comprising the step of administering to the subject an additional agent.

23. The method of claim 22, wherein the additional agent is an inhibitor of ASIC1a.

24. The method of claim 22, wherein the additional agent is an inhibitor of ASIC3.
